SEQUENCE LISTING (1 sequence)

SEQ ID NO: 1 - protein, 84 aa, homo sapiens
SVSEIQLMHNLGKHLNSMERVEWLRKKLQDVHNFVALGAPLAPRDAGSQRPRKKEDNVLVESHEKSLGEADKADVNVLTKAKSQ

(19) United States
(12) Patent Application Publication
(10) Pub. No.: US 2009/0305965 A1
(43) Pub. Date: Dec. 10, 2009

(54) Stabilized Parathyroid Hormone Composition Comprising Parathyroid Hormone, Buffer and Stabilizing Agent

(76) Inventors: Kwan-Yub Kang, Yongin-si (KR); Doo-Hong Park, Seoul (KR); Jung-Won Jeon, Seoul (KR); Yong-Seok Lee, Seoul (KR)

Correspondence Address: SUGHRUE MION, PLLC, 2100 PENNSYLVANIA AVENUE, N.W., SUITE 800, WASHINGTON, DC, 20037, US

(21) Appl. No.: 11/915,907

(22) PCT Filed: Jun. 5, 2006
(86) PCT No.: PCT/KR06/02167

§ 371 Date: Nov. 29, 2007

(30) Foreign Application Priority Data
Jun. 3, 2005 (KR) 10-2005-0047668

(51) Int. Cl. A61K 38/22; A61P 5/18

(52) U.S. Cl. 514/12

(57) ABSTRACT

Disclosed relates to a stabilized parathyroid hormone (PTH) comprising a buffer and a stabilizing agent and, more particularly, to a stabilized PTH composition in which succinic acid, malic acid, histidine or ammonium bicarbonate is used as the buffer and sorbitol or mannitol is used as the stabilizing agent. The PTH composition of the present invention can be used to formulate stably PTH protein that is much more unstable to be readily decomposed than normal low molecular weight drugs.

### TECHNICAL FIELD

The present invention relates to a stabilized parathyroid hormone (PTH) comprising a buffer and a stabilizing agent and, more particularly, to a stabilized PTH composition in which succinic acid, malic acid, histidine or ammonium bicarbonate is used as the buffer and sorbitol or mannitol is used as the stabilizing agent.

### BACKGROUND ART

Human parathyroid hormone (PTH) is secreted by the parathyroid glans as a polypeptide containing 84 amino acids. It plays an important role of regulating the calcium metabolism in the body to control the bone growth and concentration. It has been reported that PTH secreted under the low concentration of Ca²⁺ promotes the release of Ca²⁺ by acting on bone and kidney cells (Mayer, G. P. (1979) Endocrinology 2, 607-611; Rotts, J. T., Kronenberg, H. M., Rosenbaltt, M. (1982) Adv. Protein Chem. 35, 323-396) and PTH shows its biological activity even with a portion of the overall amino acid sequence, among them, PTH(1-34) is a representative active fraction containing 34 amino acids in the region of amino-terminus (N-terminus) (Br. Med. J. 1980 280:1340-44). Although the biological activities of PTH(1-34) and PTH(1-38) are similar to each other, it was found that in male and female rats, PTH(1-34) caused a side effect of an increase in the incidence of osteosarcoma (malignant bone tumor) that was dependent on dose and treatment duration (Barbehenn E K et al., Trends Endocrinol Metab. 2001 November; 12(9):383), which has been a serious trouble.

According as the genetic engineering techniques have been developed, PTH has been prepared in the form of recombinant protein from various kinds of bacteria, enzyme, etc. (J. Biol. Chem. 1989 264(8):4367-74), however, its activity may be readily lost due to the chemical denaturations, such as oxidization, deamidation and the like, and the peptide bond breaks. Experimentally, when reacting PTH(1-84) containing 84 amino acids with an oxidizing agent such as hydrogen peroxide, the activity of PTH(1-84) is rapidly reduced, which results from oxidization of residues of Met 8 and Met 18, revealed in initial researches (The Journal of Biological Chemistry, Vol. 259, No. 9, 5507-5513, 1984).

In developing a peptide having biological activity such as PTH used for the medicinal purpose, it is inevitable to consider the stability of PTH in separation and keeping. However, such instability of PTH as described above has been a serious obstacle to the formulation of PTH. Accordingly, in view of the formulation, various researches on stabilized compositions that overcome such obstacle have attracted scientific attentions.

PCT Publication No. WO/1993/11785 has disclosed a stabilized parathyroid hormone composition containing sugar and sodium chloride and PCT Publication No. WO/1999/31137 has disclosed stable crystalline forms of PTH and methods of preparation; however, both literatures do not describe the stabilized PTH composition comprising a buffer and a stabilizing agent.

Meanwhile, PCT Publication No. WO/1999/39337 has disclosed a stabilized PTH composition containing acetate or tartrate and sugars like the PTH composition containing a buffer and a stabilizing agent in accordance with the present invention. However, the ingredients of the buffer and stabilizing agent are different from those of the present invention. Moreover, the PTH composition of the invention is not decomposed more easily than that of the above referenced literature (See Table 3) and the stability of the PTH composition of the invention is increased far superior after lyophilization, thus ensuring the stability higher than that of the above literature. Furthermore, the above literature focuses on the stabilization of PTH(1-34), a portion of PTH, whereas, the present invention shows that the stabilization of full-length PTH(1-84) can be kept at a very high level, which is a distinctive feature of the present invention.

In general, it is difficult to formulate the unstable full-length PTH, since the longer the length of amino acid becomes, the easier the decomposition occurs. Besides, since it has been reported that PTH(1-34) has a side effect that may cause the incidence of osteosarcoma when administrated for a long term (FDA had instructed the pharmaceutical company to insert such risks as a warning in Forteo, the product of PTH(1-34)). Pharmaceutic companies have tried to develop the full-length PTH formulations having no risks.

Accordingly, the inventors of the present invention have learned that the PTH composition of the invention can be formulated stably from protein PTH that is more unstable than normal low molecular weight drugs since the PTH composition of the invention is not decomposed easily when succinic acid, malic acid, histidine or ammonium bicarbonate is used as the buffer and sorbitol or mannitol is used as the stabilizing agent and completed the present invention.

### DISCLOSURE OF INVENTION

### Technical Problem

It is an object of the present invention to provide a liquid parathyroid hormone composition comprising parathyroid hormone, a buffer and a stabilizing agent.

It is another object of the present invention to provide a parathyroid hormone composition lyophilized having water content below 2% and comprising parathyroid hormone, a buffer and a stabilizing agent.

### Technical Solution

The present invention provides a liquid parathyroid hormone composition comprising parathyroid hormone of a therapeutic effective dose, a buffer of a dose that can regulate pH value in a range of 4.0 to 6.0 and a stabilizing agent in a range of 0.05 to 20 parts by weight.

In addition, the present invention provides a parathyroid hormone composition lyophilized having water content below 2% and comprising parathyroid hormone, a buffer and a stabilizing agent.

Moreover, the present invention provides a method of preparing an injection using a lyophilized composition.

Hereinafter, the present invention will not be described in detail.

First, the present invention provides a parathyroid hormone comprising parathyroid hormone of a therapeutic effective dose, a buffer of a dose that can regulate pH value in a range of 4.0 to 6.0 and a stabilizing agent in a range of 0.05 to 20 parts by weight.

Human parathyroid hormone (PTH) is secreted by the parathyroid glans as a polypeptide containing 84 amino acids. It plays an important role of regulating the calcium metabolism in the body to control the bone growth and concentration. It has been reported that PTH shows its biological activity even by a portion of the overall amino acid sequence and PTH(1-34) is a representative active fraction containing 34 amino acids in the region of amino-terminus (N-terminus) (Br. Med. J. 1980 280:1340-44). In accordance with the present invention, PTH includes all fractions having PTH activities derivated from the above amino acid sequences as well as PTH having amino acid sequences of 1 to 84. In addition, PTH of the invention includes N-terminus residues of initial 34 or more, for example, PTH(1-34), PTH(1-37), PTH(1-38) and PTH(1-41), and 1 to 5 amino acid substituents for improving the PTH stability and the half-life. For example, it includes a PTH amino acid substituent that substitutes leucine or other hydrophobic amino acids, which improves the PTH stability for oxidization, for methionine residues at 8^{th} and/or 18^{th} position(s), and substitutes trypsin-nonsensitive amino acids, which improve the PTH stability for protease, e.g., histidine or other amino acids, for amino acids of 25^{th} to 27^{th} regions. Preferably, the present invention is directed to the PTH composed of 84 amino acids prepared via a method of recombinant preparation using microorganisms (U.S. Pat. No. 5,010,010) or via a chemical synthesis (U.S. Pat. No. 4,427,827).

PTH is readily decomposed due to the chemical denaturations, such as oxidization, deamidation, etc., and the peptide bond breaks. The full-length PTH(1-84) is much readily decomposed since the length is most long. Accordingly, it is most important to stabilize PTH in order to be utilized for the medicinal purpose. The inventors of the present invention have examined which composition is most stable by preparing various PTH compositions mixed with a variety of buffers and stabilizing agents.

First, to select a pH value suitable for preparing a stabilized PTH composition using PTH(1-84) (SEQ. ID. No. 1) prepared from recombinant E. coli, representative buffer solutions by pHs were prepared and PTH(1-84) was added to the solutions to compare the stabilities of the compositions. In result, it was confirmed that the pH value of the most stable PTH composition was 5.0 (See Table 1). The comparison of the stabilities was carried out via a method of measuring the amount of surviving PTH compositions using reversed phase (RP) HPLC.

According to the above results, the inventors of the present invention intended to select an appropriate buffer by setting the pH value to 5.0 and varying the kind of buffers applied to. Test PTH(1-84) compositions using succine acid, malic acid, histidine, acetic acid, glycine or citric acid as the buffer were kept at 50° C. for seven days and, then, the amounts of surviving PTH(1-84) were measured using RP HPLC. As a result, it was confirmed that the kind of buffers for preparing stabilized PTH compositions included succine acid, malic acid, acetic acid, citric acid or their salts, or amino acids of histidine, arginine or glycine, preferably, succine acid, malic acid, histidine or their salts(See Table 2).

According to the above results, the inventors of the present invention intended to select an appropriate stabilizing agent by choosing succine acid, malic acid or histidine as the buffer, highly ranked three substances that induced much amounts of surviving PTH, and varying the kind of stabilizing agents applied to. Test PTH(1-84) compositions using sorbitol or mannitol as the stabilizing agent were kept at 40° C. for seven days and, then, the amounts of surviving PTH(1-84) were measured using RP HPLC. As a result, it was confirmed that the kind of stabilizing agents for preparing stabilized PTH compositions included sorbitol, mannitol, trehalose, sucrose, EDTA or tween 80, preferably, sorbitol or mannitol (See Table 3).

When preparing a liquid PTH composition, PTH concentration was 10□/ml to 5,000□/mL, preferably, 50□/mL to 500□/mL, and included additionally a parenterally acceptable preservative, preferably, m-cresol or benzyl alcohol.

Moreover, the present invention provides a liquid parathyroid hormone composition comprising parathyroid hormone of a therapeutic effective dose, a buffer of a dose that can regulate the pH values in a range of 4.0 to 6.0 and a stabilizing agent in a range of 0.05 to 20 parts by weight.

According to the above results, the inventors of the present invention prepared a liquid PTH composition comprising a buffer and a stabilizing agent that were determined most suitable for preparing a stabilized PTH composition, prepared additionally a liquid PTH composition comprising ammonium bicarbonate as the buffer and sorbitol or mannitol as the stabilizing agent, lyophilized the liquid PTH compositions having water content below 2% and, then, kept them at 4° C. Here, since the ammonium bicarbonate used as the buffer might be volatilized under acidic conditions, the pH values of the liquid PTH composition were set at 7.0 to 8.5 and lyophilized the liquid PTH composition. The lyophilized PTH composition might be prepared as an injection via a hydration process. If the buffer added during the lyophilization was succine acid, malic acid or histidine, the hydration was carried out using stilled water, whereas, if the buffer added during the lyophilization was ammonium bicarbonate, the hydration was performed using a buffer solution, since the ammonium bicarbonate might be volatilized during the lyophilization.

The concentrations of the buffer and stabilizing agent in the lyophilized PTH are expressed as the final concentrations of liquid injections. The ingredients of the final liquid prepared for injection administration from the lyophilized composition by adding water, buffer or a mixed liquid (of buffer and stabilizing agent), are 10□/mL to 5,000□/mL of the PTH, preferably, 50□/mL to 500□/mL; 0.1 mM to 100 mM of the buffer; and 0.05 to 20 parts by weight of the stabilizing agent, the final pH value is preferably in a range of 4.0 to 6.0.

The liquid compositions prepared in the above manner was kept at 50° C. for three days to measure the amounts of surviving PTH using RP HPLC. As a result, it was confirmed that the lyophilized PTH composition was very stable as the amount of surviving PTH composition hydrated by stilled water after lyophilization was measured 80% or more (See Table 4). In particular, it was understood that the amount of surviving PTH, in which ammonium bicarbonate was used, was very stable as measured 90% or more (See Table 4).

In addition, the PTH composition of the present invention includes a parenterally acceptable preservative, preferably, m-cresol or benzyl alcohol.

The composition of the present invention may include at least one effective ingredient that provides the same or similar function in addition to the above ingredients.

The composition of the invention may contain at least one pharmaceutically acceptable carrier besides the above-described ingredients. The pharmaceutically acceptable carrier may contain at least one selected from the group consisting of saline solution, sterile water, ringer solution, buffered saline solution, dextrose solution, maltodextrose solution, glycerol, ethanol, liposome and a mixture thereof, and further contain the other ordinary additives such as antioxidant, buffer solution, bacteriostatic agent and the like, if necessary. Moreover, diluent, dispersing agent, surface-active agent and lubricant may be added thereto for preparing injectable formulations such as aqueous solution, suspension, emulsion, etc. Furthermore, a specific antibody for a target organ or other ligands may be linked to PTH in order to act on the target specifically. In addition, a chemical conjugate may be bonded to PTH(1-84) or a polymer may be mixed with PTH(1-84). For example, the PTH chemical conjugate material or the polymer mixture may include a PTH conjugated material in which PTH is chemically bonded with polyethyleneglycol, polyvinylalcohol, etc., or microparticles mixed with polylactic-co-glycolic alcohol (PLGA).

The administration methods of the PTH composition in accordance with the present invention are not limited specifically to the above, whereas, a parenteral administration (e.g., intravenous, hypodermic, intraperitoneal or topical administration) or an oral administration is available pursuant to the method desired. The parenteral administration is desired and, more particularly, the administration via hypodermic injection or intravenous injection is preferable. Dosages may be diversified pursuant to a patient s weight, age, sex, state of health and diet, administration time, administration method, excretion rate, severity of disease, etc. Daily dosage is about 0.1 μg/kg to 2 mg/kg, preferably, 0.5 μg/kg to 100 μg/kg. It is most desirable to administrate the PTH composition once or dividedly several times per day.

Having conducted a toxicity experiment by administrating the PTH composition of the present invention to mice via intravenous injections, the PTH composition was judged as a safe substance, of which 50% lethal dose LD₅₀ was at least 4 mg/kg.

The PTH composition of the invention may be used independently or in conjunction with any other therapeutic methods such as operation, hormone therapy, drug therapy, methods of using biological response modifiers, etc.

### BRIEF DESCRIPTION OF THE DRAWINGS

The application of the preferred embodiments of the present invention is best understood with reference to the accompanying drawings, wherein:

FIG. 1 is a graph showing the results of analyzing the PTH stabilities using high performance liquid chromatography (HPLC) after keeping PTH in a buffer solution (phosphate solution) of pH 6.0 to 8.0 at 50° C. for seven days, wherein 20 mM and pH 6.0 of phosphate solution, and 20 mM and pH 7.0 of phosphate solution, 20 mM and pH 8.0 of phosphate solution and initial state of standard PTH(1-84) were used, respectively;

FIG. 2 is a graph showing the result of analyzing the PTH stabilities using HPLC after keeping PTH in a buffer solution (citrate solution) of pH 4.0 to 6.0 at 50° C. for seven days, wherein 20 mM and pH 4.0 of citrate solution, 20 mM and pH 5.0 of citrate solution, 20 mM and pH 6.0 of citrate solution and initial state of standard PTH(1-84) were used, respectively;

FIG. 3 is a graph showing the results of analyzing the PTH stabilities using HPLC after keeping PTH in a buffer solution (succine acid, malic acid or citric acid) at 50° C. for seven days, wherein 20 mM and pH 5.0 of sodium citrate buffer solution, 20 mM and pH 5.0 of sodium malate buffer solution, 20 mM and pH 5.0 of sodium succinate buffer solution, and initial state of standard PTH(1-84) were used, respectively;

FIG. 4 shows graphs depicting the results of analyzing the PTH stabilities using HPLC, after keeping liquid PTH compositions comprising a buffer and a stabilizing agent at 40° C. for seven days, wherein a liquid composition containing succine acid and sorbitol was analyzed in graph (a); a liquid composition containing succine acid and trehalose was analyzed in graph (b); a liquid composition containing histidine and sorbitol was analyzed in graph (c); and a liquid composition containing histidine and trehalose was analyzed in graph (d), and, wherein 0 denotes initial state of PTH(1-84); 1 denotes that each liquid composition was kept at 40° C. for a day; 3 denotes that each liquid composition was kept at 40° C. for three days; and 7 denotes that each liquid composition was kept at 40° C. for seven days;

FIG. 5 shows graphs depicting the results of analyzing the PTH stabilities using HPLC, after hydrating lyophilized PTH compositions comprising a buffer and a stabilizing agent and keeping them at 50° C. for three days, wherein distilled water was used for the hydration, wherein a lyophilized composition containing citric acid and sorbitol was analyzed in graph (a); a lyophilized composition containing succine acid and sorbitol was analyzed in graph (b); a lyophilized composition containing malic acid and sorbitol was analyzed in graph (c); and a lyophilized composition containing histidine and sorbitol was analyzed in graph (d), and, wherein O-day denotes initial state of PTH(1-84) after hydration; and 3-day denotes that each lyophilized composition PTH(1-84) was kept at 50° C. for three days after hydration; and

FIG. 6 shows graphs depicting the results of analyzing the PTH stabilities using HPLC, after hydrating lyophilized PTH compositions comprising a volatile buffer and a stabilizing agent with a liquid containing a buffer and keeping them at 50° C. for three days, wherein lyophilized compositions containing ammonium bicarbonate and mannitol were used, wherein a citric acid solution was used for the hydration and shown in graph (a); a succine acid solution was used for the hydration and shown in graph (b); a malic acid solution was used for the hydration and shown in graph (c); and a histidine solution was used for the hydration and shown in graph (d), and, wherein 0-day denotes initial state of PTH(1-84) after hydration; and 3-day denotes that each PTH(1-84) composition was kept at 50° C. for three days after hydration.

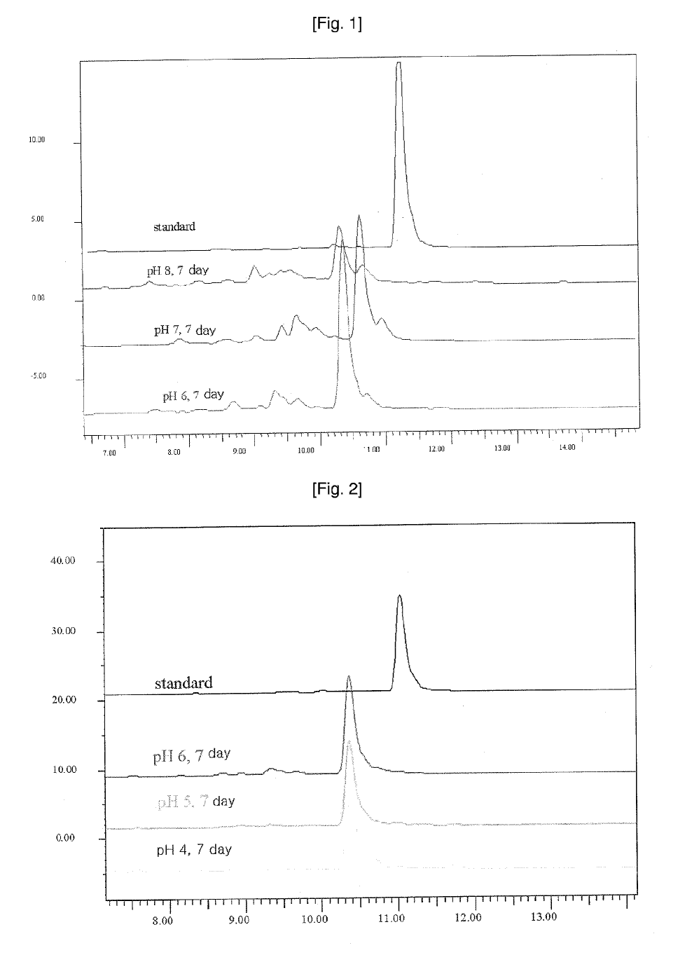

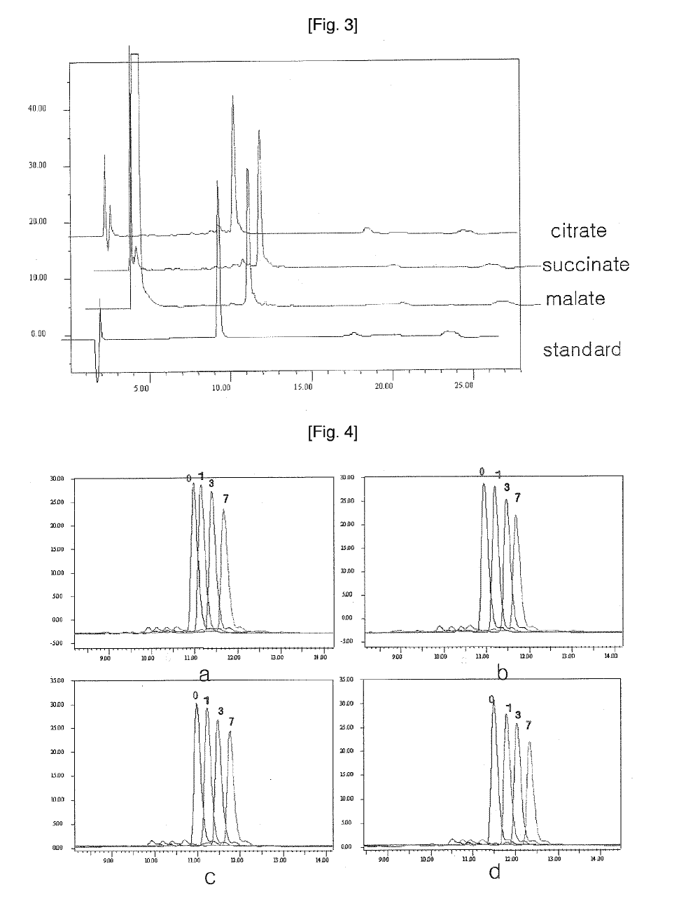

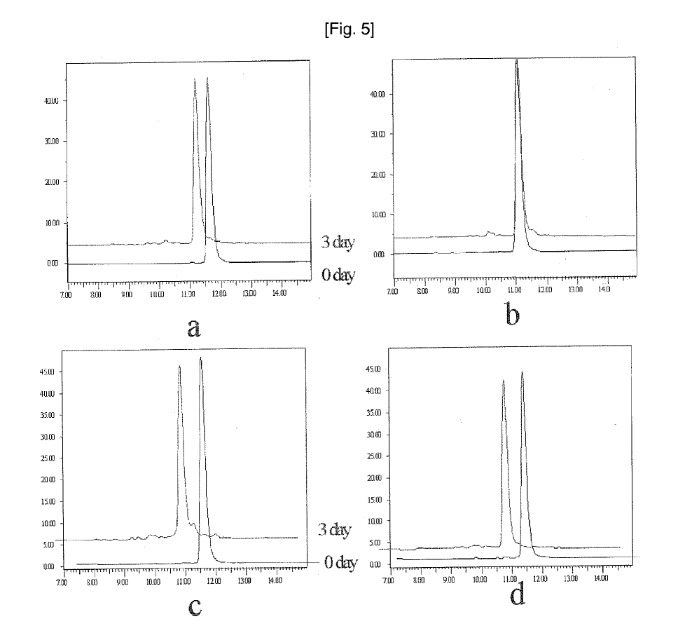

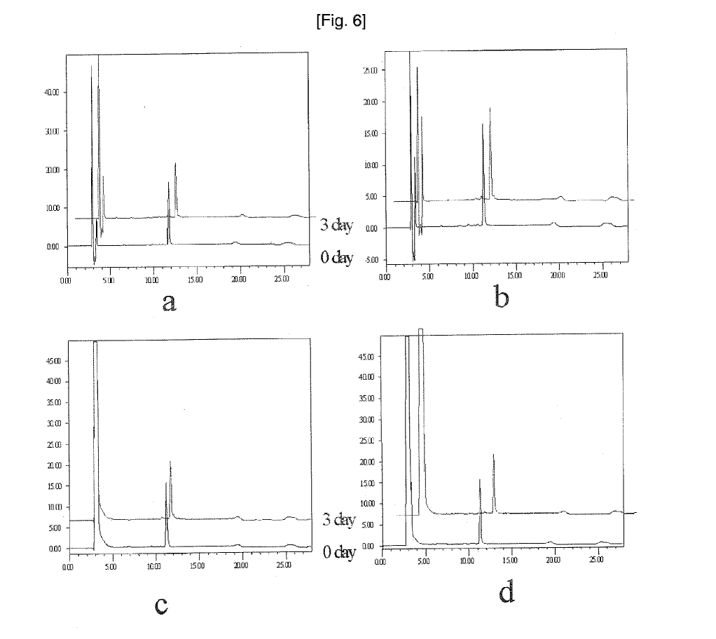

### BEST MODE FOR CARRYING OUT THE INVENTION

Hereinafter, a detail description of the present invention will be given with reference to the attached drawings. The present invention is not restricted to the following embodiments, and many variations are possible within the spirit and scope of the present invention. The embodiments of the present invention are provided in order to more completely explain the present invention to anyone skilled in the art.

### Example 1

### Selection of a pH Value Suitable for Preparing a Stabilized PTH(1-84) Composition

PTH(1-84) (SEQ. ID. No. 1) used in the present invention was prepared from recombinant E. coli. The present invention used the PTH(1-84) purely isolated from E. coli MC1061 transformed with the expression vectors (pA15UP, p153PTH and pm153PTH) via a method disclosed in Korean Patent No. 10-0230578, and expressed via a method of DO-stat fed-batch culture disclosed in Korean Patent No. 10-0255270.

More particularly, the PTH (1-84) was expressed to inclusion bodies in E. coli in the form of a fusion protein composed of phosphoribulokinase fragment and PTH(1-84) (Phosphoribulokinase fragment, which is a amino terminal fragment of fusion protein, is linked to PTH(1-84) via urokinase cleavage site). Expression induced cells were subjected to cell lysis to collect inclusion bodies. Subsequently, after dissolving the collected inclusion bodies in urea, urea was removed via a gelfiltration using dialysis or Sephadex G25(Sigma) to refold the fusion protein. The fusion protein was processed with urokinase in an optimum ratio (fusion protein:urokinase=100:1) to remove the phosphoribulokinase fragment and, then, the PTH(1-84) was purely isolated using cation exchange chromatography and reversed phase chromatography.

To select a pH value of the stabilized PTH composition, representative buffer solutions by pHs were prepared and added so as to turn the PTH(1-84) into 100 ug/ml and, then, kept 50° C. for seven days. The stabilities of the compositions by pHs were compared by analyzing the amounts of surviving intact PTH(1-84) using RP HPLC.

The RP HPLC analysis conditions were as follows: C18 HPLC column (0.46×25 cm) was equilibrated with 35% acetonitrile buffer solution containing 0.1% of TFA, the objective compositions to be analyzed were injected therein and eluted by increasing the ratio of acetonitrile gradually up to 45%. Here, the absorbance was measured at 214 nm and the flow velocity was 0.8 ml/min. The amounts of surviving intact PTH(1-84) were shown with the peak areas % for the respective test solutions based on 100% of the initial PTH peak area.

Table 1 is the results of the experiment showing the kinds and concentrations of the tested buffers and the amounts of surviving intact PTH(1-84) after being kept for seven days.

TABLE 1

| PH | Stabilized PTH (%) |
|---|---|
| PH 4.0 (20 mM Citrate) | 75 |
| PH 5.0 (20 mM Citrate) | 84 |
| PH 6.0 (20 mM Citrate) | 80 |
| PH 6.0 (20 mM Phosphate) | 75 |
| PH 7.0 (20 mM Phosphate) | 50 |
| PH 8.0 (20 mM Phosphate) | 20 |

Based on the above test results, it was determined that the pH value suitable for preparing a stabilized PTH composition was 5.0.

### Example 2

### Selection of a Buffer Suitable for Preparing a Stabilized PTH(1-84) Composition

Based on the above results of Example 1, the pH value was set at 5.0 to select an appropriate buffer by varying the kind of the buffers applied to. Here, the concentration of the liquid PTH(1-84) was 100□/ml, the kinds and the concentrations of the used buffers along with the amounts of surviving intact PTH(1-84) after being kept at 50° C. for seven days were depicted in Table 2. The analyses of the stabilized PTH compositions were carried out in the same manner as Example 1.

### Comparative Example 1

Other conditions of the experiment than the kind of the buffers were identical with those of Example 2.

TABLE 2

| | | Stabilized |
|---|---|---|
| Buffer | | PTH (%) |
| Example 2 | 20 mM Succinate (pH 5.0) | 84 |
| | 20 mM Malate (pH 5.0) | 84 |
| | 20 mM Histidine (pH 5.0) | 82 |
| Comparative | 20 mM Acetate (pH 5.0) | 70 |
| Example 1 | 20 mM Glycine (pH 5.0) | 58 |
| | 20 mM Citrate (pH 5.0) | 80 |

Based on the above test results, it was determined that the buffer suitable for preparing a stabilized PTH composition was succine acid, malic acid or histidine.

### Example 3

### Selection of a Stabilizing Agent Suitable for Preparing a Stabilized PTH(1-84) Composition

Based on the above results of Example 2, the kind of the buffers was fixed to succine acid, malic acid or histidine to select an appropriate stabilizing agent by varying the kind of the stabilizing agents applied to. Here, the concentration of the liquid PTH(1-84) was 100□/ml, the kinds and the concentrations of the used stabilizing agents along with the amounts of surviving intact PTH(1-84) after being kept at 40° C. for seven days were depicted in Table 3. The analyses of the stabilized PTH compositions were carried out in the same manner as Example 1.

### Comparative Example 2

Other conditions of the experiment than the kind of the stabilizing agents were identical with those of Example 3.

### Comparative Example 3

Other conditions of the experiment than the kind of the buffers were identical with those of Example 3.

TABLE 3

| | | Stabilizing | Stabilized |
|---|---|---|---|
| Buffer | | Agent | PTH (%) |
| Example 3 | 20 mM Sodium | 5% (w/v) | 89 |
| | Succinate (pH 5.0) | Sorbitol | |
| | 20 mM Sodium | 5% (w/v) | 81 |
| | Succinate (pH 5.0) | Mannitol | |
| | 20 mM Sodium | 5% (w/v) | 88 |
| | Malate (pH 5.0) | Sorbitol | |
| | 20 mM Sodium | 5% (w/v) | 78 |
| | Malate (pH 5.0) | Mannitol | |
| | 20 mM Histidine | 5% (w/v) | 86 |
| | (pH 5.0) | Sorbitol | |
| | 20 mM Histidine | 5% (w/v) | 75 |
| | (pH 5.0) | Mannitol | |
| Comparative | 20 mM Sodium | 0.005% (w/v) | 48 |
| Example 2 | Succinate (pH 5.0) | Tween80 | |
| | 20 mM Sodium | 5% (w/v) | 79 |
| | Malate (pH 5.0) | Trehalose | |
| | 20 mM Sodium | 0.005% (w/v) | 63 |
| | Malate (pH 5.0) | Tween80 | |
| | 20 mM Histidine | 5% (w/v) | 70 |
| | (pH 5.0) | Trehalose | |
| | 20 mM Histidine | 0.005% (w/v) | 59 |
| | (pH 5.0) | Tween80 | |
| Comparative | 20 mM Citrate | 5% (w/v) | 78 |
| Example 3 | (pH 5.0) | Mannitol | |
| | 20 mM Citrate | 5% (w/v) | 75 |
| | (pH 5.0) | Sorbitol | |

Based on the above test results, it was determined that the stabilizing agent suitable for preparing a stabilized PTH composition was sorbitol or mannitol. In addition, from the above test results, it was confirmed that the PTH composition of the present invention has a higher efficacy than that of using citrate as the buffer.

### Example 4

### Confirmation of Stability of the PTH Composition Hydrated by Stilled Water After Lyophilization

Liquid PTH(1-84) compositions comprising PTH(1-84) of 100□/ml, a buffer and a stabilizing agent or liquid PTH(1-84) compositions comprising PTH(1-84) of 100□/ml, ammonium bicarbonate and a stabilizing agent were kept at 4° C. after lyophilization. The lyophilized compositions were hydrated by stilled water or buffer solutions for injection administration and kept at 50° C. for three days. Subsequently, the stabilities of the compositions were measured.

Table 4 is the results of the experiment showing the ingredients of the lyophilized PTH(1-84) and the amounts of surviving intact PTH(1-84). The analyses of the stabilized PTH compositions were carried out in the same manner as Example 1.

### Comparative Example 4

Other conditions of the experiment than the kind of the buffers were identical with those of Example 4.

TABLE 4

| | | | Liquid | |
|---|---|---|---|---|
| Ingredients of Liquid PTH Lyophilized | | | Composition | |
| | | Stabilizing | Added for | Stabilized |
| Buffer | | Agent | Hydration | PTH (%) |
| Example 4 | 20 mM Succinate | 5% (w/v) | Stilled | 94 |
| | (pH 5.0) | Sorbitol | Water | |
| | 20 mM Histidine | 5% (w/v) | Stilled | 91 |
| | (pH 5.0) | Sorbitol | Water | |
| | 20 mM Malate | 5% (w/v) | Stilled | 90 |
| | (pH 5.0) | Sorbitol | Water | |
| | 10 mM Ammonium | 5% (w/v) | 20 mM | 93 |
| | Bicarbonate | Sorbitol | Succinate | |
| | | | (pH 5.0) | |
| | 10 mM Ammonium | 5% (w/v) | 20 mM | 93 |
| | Bicarbonate | Sorbitol | Malate | |
| | | | (pH 5.0) | |
| | 10 mM Ammonium | 5% (w/v) | 20 mM | 92 |
| | Bicarbonate | Sorbitol | Histidine | |
| | | | (pH 5.0) | |
| | 10 mM Ammonium | 5% (w/v) | 20 mM | 92 |
| | Bicarbonate | Mannitol | Succinate | |
| | | | (pH 5.0) | |
| | 10 mM Ammonium | 5% (w/v) | 20 mM | 91 |
| | Bicarbonate | Mannitol | Malate | |
| | | | (pH 5.0) | |
| | 10 mM Ammonium | 5% (w/v) | 20 mM | 91 |
| | Bicarbonate | Mannitol | Histidine | |
| | | | (pH 5.0) | |
| Comprative | 20 mM Citrate | 5% (w/v) | Stilled | 89 |
| Example 4 | (pH 5.0) | Sorbitol | Water | |

As shown in Table 4, it was understood that the PTH composition lyophilized and hydrated by stilled water in accordance with the present invention was very stable as the surviving PTH measured was 90% or more and the PTH composition containing ammonium bicarbonate as the buffer and sorbitol or mannitol as the stabilizing agent, lyophilized and hydrated by a buffer solution containing succine acid, malic acid or histidine, was very stable as the surviving PTH measured was 90% or more. Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

### INDUSTRIAL APPLICABILITY

A stabilized parathyroid hormone (PTH) composition comprising a buffer and a stabilizing agent in accordance with the present invention is formulated stably from full-length PTH(1-84) having much chemical denaturations and, more particularly, the lyophilized composition comprising ammonium bicarbonate volatilized during lyophilization has an excellent stability even after hydration, thus being usefully applied to as a stable PTH medicine.

1. A liquid parathyroid hormone composition comprising:
parathyroid hormone of a therapeutic effective dose;
a buffer of a dose that can regulate pH value in a range of 4.0 to 6.0; and
a stabilizing agent in a range of 0.05 to 20 parts by weight.

2. The liquid parathyroid hormone composition as recited in claim 1,
wherein the buffer is one selected from the group consisting of histidine, succine acid, malic acid and their salts.

3. The liquid parathyroid hormone composition as recited in claim 1,
wherein the stabilizing agent is sorbitol or mannitol.

4. The liquid parathyroid hormone composition as recited in claim 1,
wherein the buffer is succine acid and the stabilizing agent is sorbitol.

5. The liquid parathyroid hormone composition as recited in claim 1,
wherein the buffer is malic acid and the stabilizing agent is sorbitol.

6. The liquid parathyroid hormone composition as recited in claim 1,
wherein the buffer is histidine and the stabilizing agent is sorbitol.

7. The liquid parathyroid hormone composition as recited in claim 1 further comprising a parenterally acceptable preservative.

8. The liquid parathyroid hormone composition as recited in claim 7,
wherein the preservative is m-cresol or benzyl alcohol.

9. A parathyroid hormone composition, lyophilized having water content below 2%, comprising:
parathyroid hormone of a therapeutic effective dose;
a buffer of a dose that can regulate pH value in a range of 4.0 to 8.5; and
a stabilizing agent in a range of 0.05 to 20 parts by weight.

10. The lyophilized parathyroid hormone composition as recited in claim 9,
wherein the buffer is one selected from the group consisting of histidine, succine acid, malic acid, ammonium bicarbonate and their salts.

11. The lyophilized parathyroid hormone composition as recited in claim 9,
wherein the stabilizing agent is sorbitol or mannitol.

12. The lyophilized parathyroid hormone composition as recited in claim 9,
wherein the buffer is succine acid and the stabilizing agent is sorbitol.

13. The lyophilized parathyroid hormone composition as recited in claim 9,
wherein the buffer is histidine and the stabilizing agent is sorbitol.

14. The lyophilized parathyroid hormone composition as recited in claim 9,
wherein the buffer is ammonium bicarbonate and the stabilizing agent is mannitol or sorbitol.

15. A method of preparing a parathyroid hormone injection by adding a buffer solution to the lyophilized parathyroid hormone composition of claim 14.

16. The method of preparing a parathyroid hormone injection as recited in claim 15,
wherein the buffer solution is one selected from the group consisting of histidine, succine acid, malic acid and their salts.
